(19) United States
(12) Patent Application Publication
(10) Pub. No.: US 2005/0196376 A1
(43) Pub. Date: Sep. 8, 2005

(54) Preparation of polymerizable compositions

(75) Inventor: Gary L. Loomis, Rancho Santa Fe, CA (US)

Correspondence Address: G. L. LOOMIS & ASSOCIATES, INC., 990 HIGHLAND DRIVE, SUITE 212Q, SOLANO BEACH, CA, 92075, US

(73) Assignee: G. L. Loomis & Associates, Inc.

(21) Appl. No.: 11/068,580

(22) Filed: Feb. 28, 2005

Related U.S. Application Data
(63) Continuation-in-part of application No. 10/793,123, filed on Mar. 3, 2004.

(51) Int. Cl. A61K 31/765; C08J 3/28

(52) U.S. Cl. 424/78.27; 522/136

(57) ABSTRACT

Processes for modifying the viscosity of medically useful polymerizable compositions are described. The processes are carried out by providing an oxygen-free fluid composition comprising one or more polymerizable monomers and subsequently irradiating the composition with a controlled dose of high-energy radiation sufficient to effect a desired viscosity increase. Compositions produced via these process are also disclosed.

This application is a continuation-in-part of U.S. application Ser. No. 10/793,123 filed Mar. 3, 2004.

### FIELD OF THE INVENTION

This invention relates generally to processes for the production of polymerizable compositions with modified viscosities. Such compositions find utility in various medical applications.

### BACKGROUND OF RELATED ART

Compositions based on polymerizable alkyl cyanoacrylates useful for both industrial and medical applications are well-known in the art. Medical applications for alkyl cyanoacrylate compositions include uses in topical application as described in U.S. Pat. No. 5,306,490 and U.S. Pat. No. 5,403,591. Other suggested medical applications include a use for inhibiting irritation arising from prosthetic devices as described in U.S. patent application Ser. No. 08/200,953 as well as a use for inhibiting skin irritation and infection due to incontinence as described in U.S. patent application Ser. No. 08/299,935. The uses of alkyl cyanoacrylate compositions in the management of small wounds is described in U.S. Pat. No. 5,417,352. U.S. Pat. No. 6,538,026 and U.S. Pat. No. 6,476,070 describe cyanoacrylate compositions useful for filling an existing space in a mammalian body, e.g., the lumen of a blood vessel, the sac of a vascular aneurysm, a space created by a transiently placed external device, a space created by a surgical procedure, or a space created by a implantation of an object such as a stent or similar device. U.S. Pat. No. 6,335,384 describes methods for embolizing blood vessels utilizing biocompatible prepolymers including, cyanoacrylates, hydroxyethyl methacrylate, silicon prepolymers, and the like. While U.S. Pat. No. 6,476,069 provides a cyanoacrylate composition useful as an embolic agent that selectively creates a total or partial blockage in the lumen of a blood vessel, duct, fistula or other body passageway.

The preferred viscosity for alkyl cyanoacrylate compositions depends largely on the intended application of the specific composition. For example, relatively low viscosities are often preferred for adhesives where the application is to be made to a large surface area. Contrarily, where the application of a cyanoacrylate adhesive composition is to be made to a specific location on the skin, higher viscosity materials are preferred to prevent running of the material to unintended locations.

A variety of viscosity modifiers have been described for use with various 2-cyanoacrylate compositions. For example, U.S. Pat. No. 3,527,841 to Wicker et al. discloses 2-cyanoacrylate adhesive compositions for both general and surgical uses containing a poly(lactic acid) viscosity modifier that is soluble, after heating, in a wide range of 2-cyanoacrylates. After addition of the poly(lactic acid), the composition is sterilized at temperatures up to 150° C. and the resulting compositions undergo a decrease in viscosity, presumably due to degradation of the thickener during the thermal sterilization process.

U.S. Pat. No. 5,665,817 to Greff et al. discloses alkyl cyanoacrylate compositions suitable for topical application to human skin. These compositions may comprise a suitable amount of a thickening agent to provide a compositional viscosity of from about 2 to 50,000 cps at 20° C. The thickening agents employed include a partial polymer of the alkyl cyanoacrylate, poly methylmethacrylate (PMMA), or other preformed polymers soluble in the alkyl cyanoacrylate composition.

U.S. Pat. No. 3,722,599 discloses compositions that combine a polymerization inhibitor, a thickener, and a plasticizer with a fluoroalkyl cyanoacrylate for use as suture replacements or as hemostats.

U.S. Pat. No. 6,538,026, U.S. Pat. No. 6,476,069 and U.S. Pat. No. 6,476,070 disclose cyanoacrylate compositions that employ low levels of purified polymers of alkyl cyanoacrylates as viscosity modifying agents.

U.S. Pat. No. 4,038,345 to O'Sullivan, et al. describes a process for producing enhanced viscosity 2-cyanoacrylate adhesives by the addition of thickening agents. The thickeners used in these compositions are thermally treated polyacrylate polymers and the process involves heating the polyacrylate thickener to a temperature between 140°-180° C. for 30 to 180 minutes and subsequently dissolving the heat-treated thickener in the 2-cyanoacrylate composition.

A thickened allyl cynoacrylate dental adhesive composition is described in U.S. Pat. No. 4,136,138 to Dombroski, et al. The thickener is added to impart desired flow properties of the composition on the tooth and to reduce the polymerization shrinkage. The thickeners are present in quantities from 3 to 15 parts by weight and the preferred thickeners are those selected from a variety of polymers, copolymers, and terpolymers selected from such groups as polyesters, polyolefins, and polyvinyls having thickening characteristics suitable for this application. Examples of these thickeners are poly(methyl methacrylate), poly(methyl acrylate-co-acrylonitrile) (60/40 weight percent), poly(ethylacrylate), poly(butyl acrylate), and poly(ethyl acrylate-co-butyl acrylate).

U.S. Pat. No. 6,386,203 to Hammerslag describes alkyl cyanoacrylate compositions with controlled viscosity achieved by the use of fumed silica as a thickening agent. However, disadvantages arise from the difficulty of producing an even dispersion of the particulate silica in the composition and in the maintenance of such a dispersion. In fact, a practical disadvantage of most known techniques for producing viscosity modified cyanoacrylate compositions for medical applications is the requirement that the thickening agent be accurately metered and then dissolved or dispersed into the cyanoacrylate, since such processes are likely to introduce contamination.

It is know that many vinyl monomers can be induced to polymerize under the influence of high energy radiation and there are cyanoacrylate compositions specifically formulated to polymerize upon exposure to UV light and such compositions are described in U.S. Pat. No. 6,433,036.

U.S. Pat. No. 3,527,224 to Rabinowitz describes adhesive compositions comprising monomeric and polymeric n-pentyl cyanoacrylates prepared by subjecting the composition to a lengthy exposure to a UV light source. Such lengthy exposures are likely to effect undesirable side reactions such as crosslinking and decomposition. By contrast, in C. Kutal, P. A. Grutsch and D. B. Yang, “A Novel Strategy for Photoinitiated Anionic Polymerization”, Macromolecules, 24, 6872-73 (1991), the authors state that ethyl cyanoacrylate is “unaffected by prolonged (24-h) irradiation with light of wavelength >350 nm”. Such reported disparities demonstrate the need for controlled processes.

Therefore, a need exists for the production of viscosity-enhanced alkyl cyanoacrylates compositions in a fast, reproducible process that eliminates or minimizes side reactions. Move specifically, a need exists for processes for the production of medically useful cyanoacrylate compositions with controlled viscosity. Such processes should negate the need for the addition of viscosity modifying additives. Furthermore, a need exists for improved cost-efficient cyanoacrylate processes for the production of such compositions. Finally, a need exists for a simple process to simultaneously thicken and sterilize such compositions for medical applications without affecting performance of the composition. The present invention is directed to meeting these and other needs.

### SUMMARY OF THE INVENTION

The present invention meets the desires expressed above by providing simple, well controlled processes to produce viscosity enhanced compositions which include a polymerizable cyanoacrylate monomer component. Desirably, the compositions produced by processes of the present invention retain the benefits and advantages of viscosity enhanced cyanoacrylate compositions produced by other processes known in the art. An important aspect of the of the present invention is to provide processes that reduce or eliminate undesired or uncontrolled side reactions by employing process times significantly shorter than those of the processes described in the art.

In one embodiment of the present invention, there is provided a method of enhancing the viscosity of a medically useful cyanoacrylate composition by providing to a quantity of the composition a precisely controlled radiation dose sufficient to effect a viscosity increase to a precise predetermined value.

In another embodiment of the present invention, there is provided a method of enhancing the viscosity of a medically useful cyanoacrylate composition by exposing to an ultraviolet radiation source an initial cyanoacrylate composition containing a photosensitizer, wherein the photosensitizer has an absorbance maximum at or near the emission maximum of the ultraviolet radiation source.

In another embodiment of the present invention, there is provided a method of simultaneously thickening and sterilizing medically useful cyanoacrylate compositions by providing an amount of the cyanoacrylate composition to a precise radiation dose sufficient to simultaneously effect the desired viscosity increase and the requisite sterility.

In another embodiment of the present invention, there is provided a process in which a completely formulated and packaged cyanoacrylate composition is simultaneously viscosity modified and terminally sterilized in a single-step by exposing said packaged cyanoacrylate composition to a precise dose of high energy radiation such as ultraviolet light under carefully controlled conditions of temperature and environment.

The present invention will be more readily appreciated by those persons of skill in the art based on a reading of the detailed description of the invention which follows and the examples presented thereafter for illustrative purposes.

### DETAILED DESCRIPTION OF THE INVENTION

The present invention provides processes for enhancing the viscosity of an initial polymerizable fluid composition comprising at least one polymerizable monomer by subjecting said initial fluid compositions to a radiation dose sufficient to produce a resulting polymerizable fluid composition with the viscosity increased to a predetermined value. Such resulting polymerizable fluid composition are useful for medical applications as well as other applications. In certain medical applications that require that the composition be delivered through a microcatheter, it is desirable that the compositions resulting from the processes of the present invention exhibit shear thinning rheological behavior. Such Theological behavior is also referred to as pseudoplastic behavior. In an ideal fluid, usually referred to as a Newtonian fluid, the viscosity is independent of the shear rate. However at lower shear rates a shear thinning fluid is more viscous than the Newtonian fluid and at higher shear rates it is less viscous.

The high degree of control offered by the processes of the present invention allows for production of compositions with viscosities conveniently tailored to the particular use of the composition. For example, in a medical application of a polymerizable alkyl cyanoacrylate composition wherein the composition is to be topically applied to a specific location on the skin, materials with viscosities up to 50,000 cps are useful in order to prevent running of the material to unintended locations. In applications wherein the polymerizable alkyl cyanoacrylate composition is to be delivered via a syringe, catheter, sprayer or other such device compositions with viscosities in the range of 5 cps to 1,000 cps are preferred and compositions with viscosities in the range of 5 cps to 100 cps are most preferred.

Polymerizable monomers useful in embodiments of the processes and compositions obtained by the processes of the present invention include 1,1-disubstituted ethylene monomers of the formula (I):
RHC═CXY (I)
wherein X and Y are each strongly electron withdrawing groups, and R is H, —CH═CH₂; or, a C1 to C4 alkyl group, provided that X and Y are each cyano groups.

Examples of polymerizable monomers within the scope of formula (I) include 2-cyanoacrylates, vinylidene cyanides, C1-C4 alkyl homologues of vinylidene cyanides, dialkyl methylene malonates, acylacrylonitriles, vinyl sulfinates and vinyl sulfonates of the formula (II):
CH₂═CX′Y′ (II)
wherein X′ is —SO₂R′ or —SO₃R′ and Y′ is —CN, —COOR′, —COCH₃, —SO₂R′ or —SO₃R′, and R′ is H or hydrocarbyl.

Examples of specific polymerizable monomers of formula (I) for use in the present invention are 2-cyanoacrylates of formula (III): 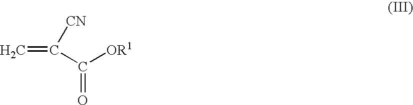
wherein R¹ is a straight-chain hydrocarbyl, a branched-chain hydrocarbyl, a cyclohydrocarbyl, a halohydrocarbyl moiety, or a substituted hydrocarbyl moiety; a group having the formula —R²—O—R³—O—R⁴ wherein R² is a 1,2-alkylene group having 2 to 10 carbon atoms, R³ is an alkylene group having 2 to 10 carbon atoms, and R⁴ is an alkyl group having 1 to 10 carbon atoms; or a group having the following formula: 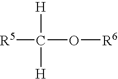
wherein R⁵ is 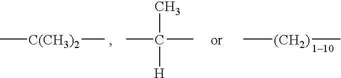
and wherein R⁶ is an organic moiety.

Specific examples of polymerizable monomers useful in the process and compositions of the present invention are alkyl 2-cyanoacrylates including ethyl 2-cyanoacrylate; n-butyl cyanoacrylate; iso-butyl 2-cyanoacrylate; n-hexyl cyanoacrylate; 2-hexyl 2-cyanoacrylate; n-octyl 2-cyanoacrylate; 2-octyl-2-cyanoacrylate; 2-ethylhexyl 2-cyanoacrylate; 3-methoxybutyl 2-cyanoacrylate; 2-butoxyethyl cyanoacrylate; 2-isopropoxyethyl 2-cyanoacrylate; and 1-methoxy-2-propyl 2-cyanoacrylate. Most preferred 2-cyanoacrylates useful in the compositions and processes of the present invention are n-butyl-2-cyanoacrylate; 2-hexyl-2-cyanoacrylate, 2-ethylhexyl 2-cyanoacrylate and 2-octyl-2-cyanoacrylate.

Also useful in certain embodiments of the present invention are polymerizable 2-cyanoacrylate monomers of formula (III) wherein R¹ is a poly(alkylene)oxide. Such poly(alkylene)oxides can include, for example, poly(ethylene)oxide, poly(propylene)oxide, poly(butylene oxide), and mixtures and copolymers thereof.

The 2-cyanoacrylates of formula (III) can be prepared according to methods known in the art. For example, U.S. Pat. Nos. 3,591,676; 3,667,472; 3,995,641; 4,035,334; and 4,650,826 the disclosures of which are each incorporated herein by reference in their entirety.

For example, the 2-cyanoacrylates can be prepared by reacting an alkyl cyanoacetate with formaldehyde in a non-aqueous organic solvent and in the presence of a basic catalyst, followed by pyrolysis of the obtained intermediate polymer in the presence of a polymerization inhibitor. The 2-cyanoacrylates monomers prepared with low moisture content and essentially free of impurities are preferred for biomedical use.

Also useful in the present invention are polymerizable 2-cyanoacrylates of formula (III) wherein R¹ is a group having the following formula: 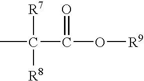
wherein R⁷ and R⁸ are hydrogen or methyl and R⁹ is an organic radical. The preparation of such cyanoacrylates are described in U.S. Pat. No. 3,995,641 the disclosures of which is incorporated herein by reference in its entirety.

Other polymerizable monomers useful in certain embodiments of the invention are 3-(acryloyloxy)sulfolanes and 3-(methacryloyloxy)sulfolanes of formula (IV): 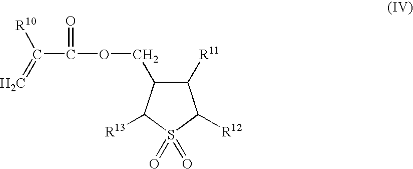
wherein R¹⁰ is H or CH₃; and wherein R¹¹, R¹², R¹³ are either H or organic moieties.

Still other polymerizable monomers useful other embodiments of the present invention are 3-(acryloyloxy)sulfolanes of the formula (V) 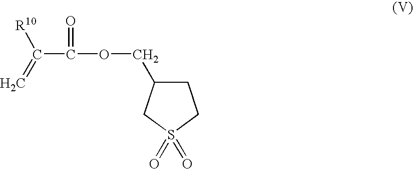
wherein X is —CN, —Cl, —Br, —I, —COCH₃, —COOR′ and R′ is H or hydrocarbyl.

In certain embodiments of the present invention the initial fluid composition is rendered essentially oxygen-free. Removal of dissolved oxygen from the initial fluid composition may be accomplished in various ways known to those skilled in the art. A common method to remove dissolved oxygen is by sparging the fluid composition with an inert gas such as nitrogen or argon. In another common method dissolved oxygen is removed by subjecting the fluid composition to repetitive freeze-pump-thaw cycles. Utilizing either method allows the reaction to be carried out in oxygen-free environment thereby ensuring an element of process control. In a particularly useful embodiment the initial fluid composition is rendered essentially oxygen-free and the process is thereafter carried out in a closed system with an atmosphere of inert gas such as nitrogen or argon maintained throughout the process.

The term high-energy radiation as used in the present invention is to be construed broadly to include any form of radiation conventionally used to initiate chemical reactions. Such radiation-induced chemical reactions include free-radical reactions, ion-radical reactions, anionic reactions, cationic reactions and concerted photochemical reactions. Non-limiting examples of such high-energy radiation include ultraviolet (UVA, 320-400 nm; UVB, 290-320 nm; and UVC, 220-290 nm); electron-beam radiation; gamma-radiation; and x-ray.

Ultraviolet radiation can be provided by any appropriate source able to generate the desired radiation, such as high pressure, medium pressure or low pressure mercury arc lamps; long wave UV lamps; He—Ne lasers; argon ion lasers; and diode pumped crystal lasers such as Nd:YAG, Nd:YVO4 or Nd:YLF.

In another embodiment the radiation source provides ultraviolet light in the range of 200 nm-600 nm. Preferably in the range 220 nm-400 nm and more preferably in the range 220 nm-300 nm. Convenient sources of suitable ultraviolet radiation are commercially available 100 to 1200 watt medium pressure, quartz, mercury-vapor lamps such as those obtainable from Hanovia Corporation, Union, N.J., USA. Ranges of wavelength output from wide-band sources such as mercury vapor lamps may be conveniently controlled by the use of suitable filters placed between the source and the compositions to be irradiated.

The most common sources of gamma-radiation are ⁶⁰Co and ¹³⁷Cs. Electron-beam irradiation involves the use of high energy electrons generated by an RF linear accelerator. Electron-beam irradiation with energy typically ranging from 3 to 10 MeV and power ranging from 1 to 50 kW is commercially available.

Certain embodiments of the present invention describe processes for enhancing the viscosity of polymerizable compositions wherein the initial fluid compositions further comprise one or more photosensitizers in a concentrations up to 5,000 ppm, preferably in concentrations of 5 to 1000 ppm, and most preferably in concentrations of 2 to 100 ppm.

The terms photosensitizer, photoinitiator, and photoactivator are often used interchangeably in the art, therefore, in the context of the present invention the term photosensitizer is to be understood to encompass materials described elsewhere as photoinitiators or photoactivators. As components of the compositions described in the present invention, photosensitizers are compounds that convert absorbed radiation into chemical energy in the form of initiating species that enhances the rates of the reactions which occur when the compositions as a whole are exposed to electromagnetic radiation such as ultraviolet light.

Photosensitizers useful in the present invention may be exemplified by benzoyl compounds; coumarin compounds; phenyl ketones such as acetophenone, benzophenone and appropriately substituted derivatives thereof; alkyl pyruvates, such as methyl, ethyl, propyl, and butyl pyruvates and appropriately substituted derivatives thereof; aryl pyruvates, such as phenyl and benzyl pyruvates and appropriately substituted derivatives thereof; benzoin ether compounds such as isobutylbenzoin ether and appropriately substituted derivatives thereof; ketal compounds such as acetophenone diethyl ketal and appropriately substituted derivatives thereof; aryl phosphine oxides and appropriately substituted derivatives thereof; and thioxanthone compounds.

Examples of photosensitizers particularly useful in the present invention include, but are not limited to, acetophenone; benzophenone; 1-hydroxycyclohexyl phenyl ketone; 1-(4-isopropylphenyl)-2-hydroxy-2-methylpropan-1-one; 2-hydroxy-2-methyl-1-phenyl-propan-1-one; 2,2-dimethoxy-2-phenyl acetophenone; 2-methyl-1-[4-(methylthio)phenyl]-2-morpholino propan-1-one; 2-benzyl-2-N,N-dimethylamino-1-(4-morpholinophenyl)-1-butanone; 2-benzyl-2-(dimethylamino)-4′-morpholinobutyrobenzophenone; 2,4,6-trimethyl-benzoyldiphenylphosphine oxide; bisacylphosphine oxide; bis-(2,6-dimethoxybenzoyl)-2,4,4-trimethylpentylphosphine oxide; bis(2,4,6-trimethyl benzoyl)phenyl phosphine oxide. Any of these may be used singly or in combination of two or more.

In another embodiment of the present invention the initial fluid composition contains a photosensitizer that is chemically bound to a non-reactive, insoluble polymer. Such a polymer-bound photosensitizer is conveniently provided in the form of particles such as insoluble beads which may be conveniently removed from the reaction medium via simple processes such as filtration, centrifugation and the like.

An important aspect of embodiments of the present invention is the provision of shortened process time in order to reduce or eliminate undesired or uncontrolled side reactions and to allow for a minimum quantity of photosensitizer compound to be used.

In another embodiment of the present invention the photosensitizer is chosen such that the wavelengths at or near the absorption maxima of the photosensitizer are matched to the wavelengths at or near the emission maxima of the ultraviolet radiation. That is, the photosensitizer is chosen such that the strong absorption bands of the photosensitizer are matched to the emmision spectrum of the radiation source. By way of example, a medium pressure mercury arc lamp has strong UV emissions between 310-320 nm while the photsensitizer 2-benzyl-2-(dimethylamino)-4-morpholinobutyro-benzophenone has strong UV absorption between 300 and 340 nm. Therefore, where a medium pressure mercury arc lamp is used as the source of radiation 2-benzyl-2-(dimethylamino)-4-morpholinobutyrobenzophenone is added to the composition as a photosensitizer. Other such combinations of photosensitizers and radiation sources will be apparent to those skilled in the art.

In other embodiments the initial solution presented is substantially free of free-radical inhibitors. Such inhibitors, which are often present in commercial polymerizable vinyl monomers such as alkyl cyanoacrylates, are conveniently reduced in concentration or are removed completely by treating the polymerizable vinyl monomer with a selective adsorbent. Such selective adsorbents for free-radical inhibitor removal are readily available from Sigma-Aldrich, Inc., St. Louis, Mo., USA (2005-2006 Catalog # 306320).

Another embodiment of the process further comprises the step of adding one or more stabilizers to the resulting fluid composition. Such stabilizers may be anionic stabilizers or free-radical stabilizers. Examples of useful anionic stabilizers include but are not limited to mineral acids such as phosphoric acids and sulfonic acids, organic acids such as acetic acid, citric acid, and Lewis acids such as sulfur dioxide and nitrogen oxides. These anionic stabilizers are useful in the resulting fluid compositions of the present invention in concentrations up to 10,000 ppm, preferably in concentrations of 5 to 1,000 ppm, and most preferably in concentrations of 10 to 200 ppm. An anionic stabilizer particularly useful for the resulting fluid compositions of the present invention is sulfur dioxide in concentration of 10 to 400 ppm.

Examples of free-radical stabilizers useful in the resulting fluid compositions of the present invention include but are not limited to hydroquinone; hydroquinone monomethyl ether (also known as 4-methoxy phenol); catechol; pyrogallol; bisphenol-A; bisphenol-S; 2,6-di-tert-butylphenol; 2,6-di-tert-butylcresol; 2,2′-methylene-bis(4-methyl-6-tert-butylphenol); 4,4′-butylidene-bis(3-methyl-6-tert-butylphenol); 4,4′-thiobis(3-methyl-6-tert-butylphenol); 2-hydroxybenzophenone; phenylsalicylic acid; 1,3,5-trimethyl-2,4,6-tris(3,5-di-tert-butyl-4-hydroxybenzyl)benzene; buytlated hydroxytoluene (also known as 3,5-di-tert-butyl-4-hydroxytoluene; methyl-di-tert-butylphenol; 2,6-di-tert-butyl-para-cresol; and BHT); butylated hydroxanisole (also known as tert-butyl-4-hydroxyanisole; (1,1-dimethylethyl)-4-methoxyphenol; tert-butyl-4-methoxyphenol; antioxyne B; and BOA). The free-radical stabilizer can also be selected from among known antioxidants, including, but not limited to, vitamin E (including alpha-tocopherol, beta-tocopherol, gamma-tocopherol, delta-tocopherol, vitamin K (including but not limited to vitamin K1 chromanol and vitamin K1 chromenol), phylloquinone, menaquinone, menadione, vitamin C, pentamethyl chromanol, non-phenolic antioxidants, octyl gallate, pentamethyl benzofuranol and derivitives thereof. Such free-radical stabilizers are most useful in concentrations of 5 to 10,000 ppm, preferably in concentrations of 5 to 1,000 ppm, and most preferably in concentrations of 5 to 750 ppm. A particularly useful stabilizer system for the resulting fluid compositions of the present invention comprises a combination of 100 to 750 ppm hydroquinone monomethyl ether, 100 to 750 ppm buytlated hydroxytoluene and 10 to 400 ppm sulfur dioxide.

In other embodiments the initial fluid composition may also include one or more agents known to produce free-radicals when suitably irradiated. Such agents are widely known as either free-radical initiators or free-radical catalysts and are useful in concentrations up to 5,000 ppm, preferably in concentrations of 1 to 1000 ppm, and most preferably in concentrations of 1 to 100 ppm. Such free-radical initiators include certain azo compounds and organic peroxides. A list of suitable azo compounds includes but is not limited to azo-bis-isobutyronitrle (also known as AIBN); 2,2′-azobis(2-methylpropionitrile); 1,1′-azobis(cyclohexanecarbonitrile) and 2,2′-azobis(2-methylbutyronitrile). Particularly useful is azo-bis-isobutyronitrle in concentrations of 1 to 100 ppm. Also useful are the azo free-radical initiators sold by E.I. DuPont commercially as VAZO™ 52, VAZO™ 64, VAZO™ 67 and VAZO™ 88. A list of suitable organic peroxides includes but is not limited to benzoyl peroxide; cumene hydroperoxide; di-tert-amyl peroxide; dicumyl peroxide; lauroyl peroxide; tert-amyl peroxybenzoate; tert-amylperoxy 2-ethylhexyl carbonate; tert-butyl peracetate; tert-butyl perbenzoate; 1,1-bis(tert-butylperoxy)cyclohexane; 1,1-bis(tert-amylperoxy)cyclohexane; 2,5-bis(tert-butylperoxy)-2,5-dimethylhexane; 1,1-bis(tert-butylperoxy)-3,3,5-trimethylcyclohexane; 2,4-pentanedione peroxide; bis(tert-butylperoxyisopropyl)benzene; ethyl 3,3-bis(tert-amylperoxy)butyrate; tert-butylperoxy 2-ethylhexyl carbonate and tert-butylperoxy isopropyl carbonate.

In another embodiment of the present invention the initial fluid composition further comprises one or more plasticizers in concentrations of 0.05% to 80% by weight of the total composition, preferably in concentrations of 5.0% to 60% % by weight of the total composition, and most preferably in concentrations of 10% to 50% by weight of the total composition.

In another embodiment of the present invention the process further comprises the step of adding one or more plasticizers to the resulting fluid composition in concentrations of 0.05% to 80% by weight of the total composition, preferably in concentrations of 5.0% to 60% % by weight of the total composition, and most preferably in concentrations of 10% to 50% by weight of the total composition.

The term plasticizer in the context of the present invention is to be construed as any material which is soluble or dispersible in a polymerizable composition, and which increases the flexibility of the polymer obtained from polymerization of said polymerizable composition. Such plasticizers should be biocompatible to the extent required for the intended application. For example, a plasticizer used in a coating on the skin surface should be compatible with the skin as measured by the lack of skin irritation and a plasticizer used for an implant in the body should be non-toxic or of a toxicity sufficiently low as to be tolerated by the body. Suitable plasticizers are well known in the art and include those disclosed in U.S. Pat. Nos. 2,784,127 and 4,444,933 the disclosures of both of which are incorporated herein by reference in their entirety.

A list of plasticizers useful in the present invention includes, but is not limited to, fatty acid esters, citrate esters, phthalate esters, benzoate esters, and certain aromatic phosphate esters. By way of example, such useful plasticizers include butyl benzyl phthalate, dibutyl phthalate, diethyl phthalate, dimethyl phthalate, dioctyl phthalate, 2-ethylhexyl phthalate, benzoate esters of di- and poly-hydroxy branched aliphatic compounds, tri(p-cresyl) phosphate, alkyl myristates and the like. Plasticizers particularly useful in this invention are acetyl triethyl citrate, acetyl tri-n-butyl citrate, acetyl tri-n-hexyl citrate, and n-butyryl tri-n-hexyl citrate.

In other embodiments of the present invention the initial fluid composition may further comprise an accelerator. In the context of the present invention an accelerator is a molecule containing a reactive carbon-carbon double bond such an allyl, vinyl, or acrylate group, that is capable of increasing the rate of a photochemical or free radical reaction. Suitable accelerators include, but are not limited to, N-vinyl pyrrolidinone, 2-vinyl pyridine, 1-vinyl imidazole, 9-vinyl carbazone, acrylic acid, and 2-allyl-2-methyl-1,3-cyclopentane dione.

In another embodiment of the present invention the process further comprises the step of adding one or more radiopaque contrast agents to the resulting polymerizable fluid composition.

In other embodiments of the present invention the initial fluid compositions further comprises one or more radiopaque contrast agents.

In use in the intended applications the radiopaque compositions thus produced allow a practitioner to visualize delivery of the fluid composition to the desired site guided by x-ray techniques such as fluoroscopy. Visualization is particularly useful when using catheter delivery techniques in order to ensure both that the polymerizable fluid composition is being delivered to the intended vascular site and that the requisite amount of the polymerizable fluid composition is delivered. Additionally, the use of contrast agents is beneficial during post-treatment procedures to visualize the embolized mass during, for example, surgery or to monitor the disease condition for re-treatment purposes.

Radiopaque contrast agents particularly useful in the present invention are insoluble contrast agents in particulate form. Examples of such insoluble, particulate contrast agents include but are not limited to tantalum, tantalum oxide and barium sulfate as well as noble metals such as gold, palladium and platinum as well as mixtures and alloys thereof. Particularly useful insoluble, particulate contrast agents are precipitated gold and gold alloy powders as well as atomized gold and gold alloy powders such as those available from Technic Inc., Woonsocket, R.I., USA. Insoluble metal-cation salts of anionic polymer such as those described in U.S. Pat. No. 5,702,682 are also useful in certain embodiments of the present invention.

In other embodiments the temperature of the reaction medium is carefully controlled throughout the course of the process. The minimum temperature maintained throughout the process should be above the temperature of thermal transitions for each components in the initial fluid compositions. These thermal transitions may include, but are not limited to, a freezing point, a glass transition temperature or a limiting solubility temperature. To avoid thermal polymerization of the one or more polymerizable monomers of the initial solution it is generally desirable to maintain the temperature of the processes of the present invention in the range of −50° C. to 50° C. This type of temperature control is conveniently achieved by use of a water-jacketed photochemical reaction vessel through which is circulated a thermostatically controlled fluid. A suitable photochemical apparatus to effect such temperature control is commercially available from Ace Glass Inc., Vineland, N.J. Such a photochemical apparatus is conveniently equipped with a means for stirring the reaction mixture. A suitabe means for stirring is a combination of a fluoropolymer-coated magnetic stir bar in the reaction medium controlled with an external magnetic stirrer. Effective stirring is particularly important for embodiments in which the initial fluid composition contains an insoluble contrast agent in particulate form.

Yet another embodiment provides a continuous process by the use of a thin film photochemical reactor such as the apparatus commercially available from Ace Glass Inc., Vineland, N.J., USA.

The following examples are presented to illustrate embodiments of the invention, and shall not be viewed as limiting the scope of the invention.

### EXAMPLES

All of the examples shown below utilize a commercial photochemical reactor assembly (available as Catalog Number 7862-245 from Ace Glass Company, Vinland, N.J., USA) consisting of a 250 ml cylindrical, 3-neck, flat-bottomed, water-jacketed reaction vessel; a circulating water chiller; a quartz immersion well into which is inserted a 450 watt medium pressure, quartz, mercury-vapor lamp. Agitation of the reaction medium is acheived with a fluoropolymer-coated magnetic stir bar in the reaction fluid controlled withan external magnetic stirrer.

### Example 1

To the reaction vessel was introduced a solution containing 294 g of n-hexyl cyanoacrylate rendered substantially free of free-radical stabilizers by passing through a 10″ X ¾″ column of absorbent (Sigma-Aldrich, Inc., St. Louis, Mo., USA; 2005-2006 Catalog Number 306320) and 149 g acetyl tri-n-butyl citrate. This solution was then degassed by sparging with argon at a flow rate of approximately 100 ml/min for 2.0 hrs after which the vessel and its contents was maintained under an argon atmosphere. The circulating water temperature was set and maintained at 20° C., stirring was commenced and the UV lamp was ignited. After 30 min. the UV lamp was extinguished and 100 ppm 4-methoxy phenol, 100 ppm hydroquinone and 25 ppm sulfur dioxide were immediately introduced into the reaction mixture. Viscosities of the initial and final compositions are measured at 25° C. with a Brookfield cone and plate viscometer. The initial viscosity was 3.9 cps and the final viscosity was 7.2 cps.

### Example 2

To each of four 3-ml borosilicate glass ampoules was added 2.0 g of stabilizer-free n-hexyl cyanoacrylate containing 20 ppm benzophenone. The filled ampoules were then blanketed with argon and flame-sealed. The sealed ampoules were suspended within the reaction vessel and positioned to be equidistant from the UV source. The circulating water temperature was set and the temperature was maintained at 15° C. The UV lamp was ignited and samples were periodically removed and the viscosity was measured at 25° C. with a Brookfield cone and plate viscometer. The results are presented in Table 1 below. These data clearly demonstrate the controlled viscosity enhancement of an alkyl cyanoacrylate afforded by this process.

TABLE 1

| Sample | Irradiation Time (min.) | Viscosity (cps) |
|---|---|---|
| 1 | 0 | 4.6 |
| 2 | 25 | 7.2 |
| 3 | 45 | 14.3 |
| 4 | 60 | 21.6 |

### Example 3

100 ml n-hexyl cyanoacrylate was rendered substantially free of free-radical stabilizers by passing through a 10″ X ¾″ column of absorbent (Sigma-Aldrich, Inc., St. Louis, Mo.; 2005-2006 Catalog # 306320) under a positive pressure of argon. This stabilizer-free n-hexyl cyanoacrylate was degassed by sparging the solution with argon at approximately 100 ml/min for 2.0 hrs and the resulting solution was maintained under an argon atmosphere. To 10.00 g of this stabilizer-free, degassed n-hexyl cyanoacrylate was added 0.05 mg 2,2′-azobis(2-methylpropionitrile) to afford a solution in which the 2,2′-azobis(2-methylpropionitrile) was 5 ppm and 3.0 g of this solution was transferred to a 15 ml polypropylene centrifuge, blanketed with argon and fitted with a gas tight screw cap. The tube was the suspended within the reaction vessel with the aid of a wire. The circulating water temperature was set and maintained at 15° C. and the UV lamp was ignited. After 30 min. the UV lamp was extinguished and the viscosities of the initial and final compositions were measured at 25° C. with a Brookfield cone and plate viscometer. Initial viscosity was measures to be 4 cps and viscosity after 30 min. was measures to be 13 cps. The solution was further irradiated for an additional 6 min. (36 min. total) and viscosity was measures to be 18 cps. To 2.0 g of the resulting reaction product was quickly added to a solution containing 1.0 g acetyl tri-n-butyl citrate in which was dissolved 750 ppm 4-methoxy phenol, 750 ppm butylated hydroxy toluene and 75 ppm sulfur dioxide. This resulting material was designated Product A and had a measured viscosity of 20 cps at 25° C. To this 1.6 g of this Product A was added 1.0 g of particulate gold (Technic 504 obtained from Technic Inc., Woonsocket, R.I., USA) to afford a composition designated Product B. Using a 3 ml syringe fitted with a 22 gaugehypodermic needle approximately 0.5 g of Product B was slowly injected into fresh bovine whole blood at 37° C. Under these conditions Product B rapidly polymerized upon contacting the blood and a continuous mass (bolus) of firm but flexible polymer composition was produced. Such bolus formation illustrates the cohesiveness or cohesive character of Product B during the polymerization reaction initiated upon contact with bovine blood.

1. A process comprising the steps of:
i. providing a substantially oxygen-free initial fluid composition comprising at least one alkyl 2-cyanoacrylate monomer, and
ii. subjecting said initial fluid composition to a dose of high-energy radiation sufficient to afford a resulting fluid composition with a viscosity higher than that of said initial fluid composition.

2. The process of claim 1 wherein said high-energy radiation is ultraviolet radiation.

3. The process of claim 2 wherein said ultraviolet radiation has a wavelength from 220 nm to 600 nm.

4. The process of claim 1 wherein said initial fluid composition further comprises a photosensitizer.

5. The process of claim 4 wherein said photosensitizer is chosen such that the wavelengths at the absorption the maxima of said photosensitizer are matched to the wavelengths at the emission maxima of said ultraviolet radiation.

6. The process of claim 1 wherein said initial fluid composition further comprises a free-radical initiator.

7. The process of claim 6 wherein said free-radical initiator is an azo compound.

8. The process of claim 7 wherein said azo compound is azo-bis-isobutyronitrle.

9. The process of claim 6 wherein said free-radical initiator is an organic peroxide.

10. The process of claim 1 wherein said initial fluid composition further comprises a plasticizer.

11. The process of claim 1 wherein said resulting fluid composition the viscosity has a viscosity from 5 to 1000 cps.

12. The process of claim 1 further comprising the step of adding one or more stabilizers to said resulting fluid composition.

13. The process of claim 12 wherein at least one of said one or more stabilizers is a free-radical stabilizer.

14. The process of claim 13 wherein said free-radical stabilizer is chosen from the group consisting of hydroquinone, hydroquinone monomethyl ether, buytlated hydroxytoluene, butylated hydroxanisole, 2,6-di-tert-butylphenol and 2,6-di-tert-butylcresol.

15. The process of claim 12 wherein at least one of said one or more stabilizers is an anionic stabilizer.

15. The process of claim 13 wherein said anionic stabilizer is sulfur dioxide.

16. The process of claim 1 further comprising the step of adding a plasticizer to said resulting fluid composition.

17. The process of claim 16 wherein said plasticizer is a citrate ester.

18. The process of claim 17 wherin said citrate ester is acetyl tri-n-butyl citrate.

19. The process of claim 1 further comprising the step of adding to said resulting fluid composition acetyl tri-n-butyl citrate in a concentration of 10% to 50% by weight of the total composition, 50 to 750 ppm 4-methoxy phenol, 50 to 750 ppm butylated hydroxy toluene and 20 to 100 ppm sulfur dioxide.

20. The composition obtained by the process of claim 1.
